(19) United States
(12) Patent Application Publication
(10) Pub. No.: US 2008/0112903 A1
(43) Pub. Date: May 15, 2008

(54) Topical Oral Care Compositions

(75) Inventor: Robert Eric Montgomery, Monterey, MA (US)

Correspondence Address: DISCUS DENTAL IMPRESSIONS, INC., 8550 HIGUERA STREET, CULVER CITY, CA, 90232, US

(73) Assignee: Britesmile Professional LLC, Culver City, CA (US)

(21) Appl. No.: 11/932,097

(22) Filed: Oct. 31, 2007

Related U.S. Application Data
(62) Division of application No. 10/056,296, filed on Jan. 24, 2002.
(60) Provisional application No. 60/263,884, filed on Jan. 24, 2001.

(51) Int. Cl. A61K 8/46

(52) U.S. Cl. 424/57

(57) ABSTRACT

Embodiments of the present invention are directed to topical oral care compositions including a free radical scavenging precursor compound.

### RELATED APPLICATION(S)

This application claims priority from U.S. Provisional Application Ser. No. 60/263,884, filed Jan. 24, 2001 hereby incorporated by reference in its entirety.

### TECHNICAL FIELD

The present invention relates to topical oral compositions that include a compound that generates a free radical scavenger compound to scavenge free radicals. Compounds according to the present invention include ascorbic acid precursor compounds that generate ascorbic acid when placed in the oral cavity. The present invention also relates to methods of using such compositions for preventing or curing various disease processes or symptoms of the oral cavity that are responsive to the presence of free radicals. The present invention also relates to oral care compositions that include phosphate precursor compounds that generate phosphates useful in oral care methods.

### BACKGROUND ART

The maintenance of healthy teeth and gums has long been the goal of modern dentistry. Since the advent of fluoride almost 50 years ago, the incidence of tooth decay, or caries, has decreased substantially. Fluoride is now found in drinking water and many consumer dental products, and has won widespread acceptance as a safe and effective ingredient that has yielded tremendous health benefits for large segments of the population. Innovation in the past 20 years has further expanded the therapeutic potential of topical oral care products. Antibacterial agents, such as triclosan, chlorhexidene salts, cetylpyridinium chloride, and domiphen bromide have been added to topical dental products to address gingivitis, periodontitis, halitosis and caries. Tooth desensitizers, such as potassium nitrate, strontium chloride and fluoride salts have been successfully employed to decrease tooth sensitivity. Tartar control agents, such as pyrophosphate salts, zinc citrate trihydrate, sodium hexametaphosphate and sodium tripolyphosphate are commonly used in toothpastes and mouthwashes to prevent the buildup of dental calculus on tooth surfaces. Tooth whitening agents, such as hydrogen peroxide, carbamide peroxide, sodium percarbonate, sodium perborate, chlorine dioxide, and sodium tripolyphosphate, have more recently been added to many dental products as auxiliary ingredients that add the perceived value of white (as well as healthy) teeth to the consumer.

The role of reactive oxygen species (ROS) in many disease processes has come to light through research into the etiology of various cancers. The term ROS encompasses free radicals (such as the hydroxyl radical, OH.) or precursors to free radicals (such as hydrogen peroxide, H₂O₂). While it is clear that the incidence of oral cancer is much higher in individuals who smoke and/or drink alcohol, and it is tempting to assume that such oral cancer is a direct result of the oxidative stress place on the soft tissues of the oral cavity, very few approaches have yet been developed to address the problem. Most efforts to date have centered on the diagnostic techniques required to detect oral cancer at early stages, when it is most curable. A number of means for measuring oxidative stress in oral soft tissues have been developed. A number of topical and oral compositions have been proposed that comprise one or more antioxidants and claim to counteract the soft tissue effects of free radicals that may be generated naturally or from environmental stress.

It has also been recently discovered that a certain type of tooth staining is the result of the formation of Maillard, or non-enzymatic browning, reaction products. Maillard reaction products are generally based on furfural derivatives, which have multiple conjugated double bond structures that absorb light in the visible region of the electromagnetic spectrum (in the range of 400 to 550 nanometers). Such an absorption spectrum results in these reaction products appearing off-white to yellowish-red to the naked eye. When Maillard reaction products form on the surface of the teeth, the teeth appear to be stained. The Maillard reaction can occur when reducing sugars (such as glucose) are present in aqueous solution together with amino-functional compounds (such as amino acids or the amino-side chains of proteins). The oral cavity and, in particular, surfaces of the teeth that are covered with dental plaque, is an ideal environment (moisture, glucose, proteins, and body heat together in one locale) for encouraging the formation of Maillard reaction products. It has been further recognized that the Maillard reaction is a free radically-induced reaction that can be catalyzed by reactive oxygen species.

While ascorbic acid is a known scavenger of ROS, and is relatively ubiquitous in biological systems, its formulation within cosmetics and toiletries has been somewhat problematical, due to the propensity of ascorbic acid to oxidize immediately in the presence of oxygen and moisture. Thus, a good deal of work has concentrated on the identification of ascorbic acid derivatives that remain stable until contact with a biological surface possessing enzymatic activity capable of releasing free ascorbic acid from the derivatized parent compound.

Ascorbyl phosphate, specifically ascorbic acid derivatized at the C-2 position with an orthophosphate, pyrophosphate, tripolyphosphate, or polyphosphate group, has been identified as a storage-stable source of biologically active ascorbic acid. Upon contact with a biological tissue surface possessing phosphatase enzyme activity (capable of scission of the carbon-oxygen bond at the C-2 position of the parent ascorbic acid molecule), ascorbyl phosphate is converted into free ascorbic acid, as well as a phosphate ion (or a pyrophosphate, tripolyphosphate, or polyphosphate ion, as the case may be).

Interestingly, it has recently been determined that a single dose of ascorbic acid administered intraperitonaeally caused an increase in serum alkaline phosphatase enzyme in Wistar rats with artificially induced periodontal lesions. The study further suggests a role for ascorbic acid as an induction signal in periodontal lesion remodeling. The study does not, however, suggest that topical application of ascorbyl phosphate would have any biological significance.

Methods of manufacturing ascorbyl phosphate and its salts are described in detail in U.S. Pat. No. 4,999,437 (Dobler, et al), U.S. Pat. No. 5,110,950 (Seib, et al), U.S. Pat. No. 5,149,829 (Seib, et al), U.S. Pat. No. 5,420,302 (Kaiser, et al), U.S. Pat. No. 6,063,937 (Dlubala, et al) and U.S. Pat. No. 6,121,464 (Bottcher, et al) each of which are hereby incorporated by reference in their entireties. A number of these references suggest the use of ascorbyl phosphate as a source of ascorbic acid in feed supplement, particularly for fish. Such applications are, by definition, means of administering ascorbic acid to an organism or subject by means of ingestion, rather than topical application to the oral cavity (whereby conversion to free ascorbic acid occurs prior to ingestion, thereby exerting a therapeutic effect on one or more tissue surfaces of the oral cavity). The notion of oral residence time, or the amount of time a composition remains in contact with the oral cavity, is of importance in differentiating the compositions and methods of the present invention from those intended for administration by feeding (where contact time is relatively short).

Considering the number of oral diseases or conditions that result directly or indirectly from the formation of ROS in the oral cavity, few attempts have been made to attenuate oxidative stress in the oral environment. The same cannot be said about efforts in non-oral skin care, where the effects of ROS on skin aging have been recognized for many years. U.S. Pat. No. 6,184,247 describes methods of enhancing the rate of mammalian skin desquamation or exfoliation that utilize compositions comprising an ascorbic acid derivative such as a salt of ascorbyl phosphate, ascorbyl sulfate, and mixtures thereof. As defined in the specification, the term skin refers to structures defined by an epidermal layer and a dermal layer, and normal turnover time of cells (period between basal cell formation and outer surface desquamation) is approximately 28 days. Whereas the compositions and methods described by the inventors increase cell turnover by as much as 23%, this increase in desquamation is greatly overshadowed by the natural turnover rates of oral mucosal tissues (hard palate, buccal mucosal, floor of the mouth mucosal), which are normally in a range from about 10% to about 100% greater than that of skin.

Topical alkyl-2-O-ascorbyl phosphates are described in U.S. Pat. Nos. 5,607,968 and 5,780,504. While displaying the aqueous stability necessary to formulate these novel ingredients within skin care compositions, it is unlikely that both the ascorbic acid and underivatized phosphate moieties would be released for immediate bioavailability if applied to the oral cavity. The oral toxicity of these derivatives and their topical formulations is also unknown.

Ascorbyl phosphate has also been employed in cosmetic and toiletry formulations, in conjunction with an iminium ion scavenger, to prevent nitrosation reactions and subsequent nitrosamine formation (See U.S. Pat. No. 5,807,542). Nitrite ions and iminium ions may be generated as a result of the combination, in the same formulation, of many different cosmetic and toiletry ingredients; however it is inappropriate to include such ingredients together within an oral composition that must, by definition, be suitable for partial ingestion by the user.

Kayane, et al (U.S. Pat. No. 5,244,651) describe a method of desensitizing hypersensitive dentin comprised of treating teeth with a colloid produced by mixing a salt of a polyvalent metal and a polyol phosphate. The useful polyvalent metals described are required to form a water-insoluble (or hardly soluble) hydroxide in order to have utility in the practice of the invention (Col 2, lines 20-23). Calcium hydroxide is not water-soluble and thus is not mentioned by the inventors as a polyvalent metal of utility in the tooth desensitizing methods of the invention.

Spaltro, et al (U.S. Pat. No. 5,202,111) describe phosphorylated polyhydroxy compounds for tartar control applications. The polyhydroxy compounds used as starting materials in the manufacture of the phosphorylated analogues are described as acyclic polyols, cyclic polyols, and higher oligosaccharides, and include sugars, sugar alcohols, modified saccharides and polysaccharides. Ascorbic acid is not specifically anticipated as a starting material, nor are the phosphorylation methods described (see Examples 1-4, Cols 5 and 6) suitable for manufacture of ascorbyl phosphate.

Thus, there is a need for oral compositions and methods comprising compounds that can scavenge free radicals and thereby reduce or eliminate the potential effects of reactive oxygen species on the tissues of the oral cavity.

There is also a need for oral compositions and methods that can provide for increased tooth surface remineralization capacity by increasing the availability of phosphate ions.

There is also a need for oral compositions and methods that can scavenge free radicals in the oral cavity, while simultaneously providing for increased tooth surface remineralization capacity by increasing the availability of phosphate ions.

There is furthermore a need for dental compositions and methods comprising an ascorbyl phosphate compound in combination with one or more additional dentally therapeutic ingredients, such as anticaries agents, tartar control agents, antiplaque agents, antimicrobial agents, antigingivitis agents, desensitizing agents, and combinations thereof.

There is also a need for dental compositions and methods that can perform the free radical scavenging benefit as described above, while simultaneously providing at least one additional therapeutic benefit for the tissues of the oral cavity.

There is also a need for aqueous dental compositions that contain a stable source of ascorbic acid that does not deteriorate in the package prior to use in the oral cavity.

There is additionally a need for stable dental compositions that provide a bioactive source of ascorbic acid upon contact with tissues of the oral cavity.

### SUMMARY OF THE INVENTION

Embodiments of the present invention are directed to compositions and methods comprising compounds that can scavenge free radicals present in the oral cavity, such as on tissue surfaces in the oral cavity, and thereby reduce or eliminate the potential effects of reactive oxygen species on the tissues of the oral cavity. The compositions of the present invention include a precursor compound that generates a free radical scavenger when placed onto the tissue surface in an oral cavity. The free radical scavenger will in turn scavenge free radicals with which it comes in contact, thereby reducing the concentration of free radicals in the oral cavity. Reducing free radical scavengers in the oral cavity reduces the effects of conditions and diseases which result from the presence of free radicals in the oral cavity.

Embodiments of the present invention are further directed to compositions and methods that can provide for increased tooth surface remineralization capacity by increasing the availability of phosphate ions. The compositions of the present invention include a phosphate precursor compound that can generate phosphate ions when placed onto the tissue surface in an oral cavity. The phosphate ions generated by the phosphate precursor compound combine with cations, such as calcium ions present in the oral cavity, to form calcium phosphates that precipitate on or within the tooth enamel or dentinal tubules, and accordingly, remineralize the tooth.

Embodiments of the present invention are directed to compositions and methods that include compounds which scavenge free radicals in the oral cavity, while simultaneously providing for increased tooth surface remineralization capacity by increasing the availability of phosphate ions.

Additional embodiments of the present invention include compositions and methods that can perform the free radical scavenging benefit as described above, while simultaneously providing at least one additional therapeutic benefit for the tissues of the oral cavity. According to the present invention the compositions optionally include one or more additional dentally therapeutic ingredients, such as anticaries agents, tartar control agents, antiplaque agents, antimicrobial agents, antigingivitis agents, desensitizing agents, and combinations thereof.

The compositions of the present invention include the ingredients in the form of an oral rinse, a mouthwash, a dentifrice with or without abrasives, toothpastes, creams, gels and other topical formulations well known to those skilled in the art.

An additional embodiment of the present invention includes aqueous dental compositions that contain a stable source of ascorbic acid that retains its potency and does not deteriorate in the package prior to use in the oral cavity. Further embodiments of the present invention provide stable dental compositions that include a bioactive source of ascorbic acid upon contact with tissues of the oral cavity.

Particular embodiments of the present invention are directed towards therapeutic dental compositions and methods that contain an ascorbyl phosphate in an orally acceptable carrier, together with an optional auxiliary therapeutic ingredient, to prevent or cure various disease processes or symptoms of the oral cavity associated with the presence of free radicals. According to this aspect of the present invention, the composition including the ascorbyl phosphate is contacted with the tissue of the oral cavity. The ascorbyl phosphate generates ascorbic acid that then scavenges free radicals present on the tissue surface within the oral cavity, thereby reducing the concentration of free radicals present in the oral cavity. A particularly preferred ascorbyl phosphate is ascorbyl-2-phosphate.

An additional embodiment of the present invention includes a method of therapeutically treating an individual afflicted with a disease or condition in the oral cavity associated with the presence of free radicals. The method includes contacting tissue within the oral cavity with a composition including a therapeutically effective amount of a free radical scavenging compound or a free radical scavenging precursor compound in a manner to reduce the concentration of free radicals within the oral cavity. According to one aspect of the present invention, the composition is contacted with the tissue within the oral cavity for a period of time sufficient to reduce the concentration of free radicals. According to certain embodiments, the composition contacts the tissue for a period of 60 minutes or less, preferably for greater than 5 minutes, or more preferably for greater than 10 minutes. The free radical scavenging compound includes ascorbic acid. The free radical scavenging precursor compound includes an ascorbyl phosphate compound. The free radical scavenging precursor compound generates a free radical scavenging compound that then scavenges free radicals present within the oral cavity.

In addition, the preferred ascorbyl phosphate compounds described herein display a surprising degree of adhesion to oral tissue including tooth enamel surface. Adhesion or attachment to the tooth enamel surface can prolong the effective duration of ascorbic acid and phosphate ion release into saliva by endemic phosphatase enzyme.

The term “topical” shall be used in this disclosure to mean applied to the surface of the intended target oral tissue, but shall also include oral tissue subsurfaces as a result of penetration of all or part of the inventive composition through said surface. For example, topical application of the inventive composition to the tooth enamel surface shall also include the underlying tooth structure (dentin, pulp, etc) that may become exposed to the composition by way of penetration through intact tooth enamel, or alternatively through the temporary exposure of such underlying oral tissue structure during a dental procedure.

These and other objects, features and advantages of the present invention will become apparent by reference to the remaining portions of the specification and the claims.

### DETAILED DESCRIPTION OF CERTAIN PREFERRED EMBODIMENTS

According to certain preferred embodiments of the present invention, a topical oral care composition is provided which includes a free radical scavenging precursor compound in combination with orally acceptable carriers and optionally, therapeutically active agents. A preferred free radical scavenging precursor compound is the family of ascorbyl phosphate compounds. The composition is placed within the oral cavity in contact with tissue within the oral cavity for a period of between about 5 minutes and about 60 minutes.

The addition of ascorbyl phosphates and their salts, such as the trisodium salt of ascorbyl-2-monophosphate, to toxicologically acceptable oral carriers results in useful oral care compositions that may be used to counteract tooth decay, prevent tooth stain accumulation and assist in the regenerative process of periodontal tissues.

Ascorbyl phosphates include those commonly understood by the term “ascorbyl phosphate” and additionally the phosphate esters of L-ascorbic acid, as well as salts thereof, described by the following formula I

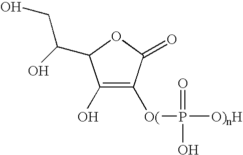

where n is between 1 and 10, preferably between 1 and 5 and more preferably between 1 and 3, and specifically n can be 1, 2, 3, 4, or 5 or higher.

Preferred salts of the above compound include the sodium, potassium, ammonium, and calcium salts of the compound. As indicated by the compound of formula I, the compounds of the invention include a phosphate group (a mono-, di-, tri-, or polyphosphate moiety) at the 2-position of the ascorbic acid molecule. While the phosphate group can be derivatized, according to one preferred aspect, the phosphate group is non-derivatized as shown in formula I, in order that biologically viable phosphate ions are immediately released into salivary solution upon contact with tissue in the oral cavity or the ascorbyl phosphate compound with phosphatase enzymes in the oral cavity. Biological viability may include participation in calcium phosphate formation and precipitation. Both the ascorbic acid and phosphate moieties of the inventive compositions become biologically viable after contact with the oral cavity.

The D-ascorbic acid form of ascorbyl phosphate may also have utility in certain oral applications, where only free radical scavenging is desired and not other biological functions (where the L-ascorbic acid is the only active form). It is to be understood to those of skill in the art that formula I represents a preferred compound according to the present invention, but that other compounds having free radical scavenging properties are well known to those of skill in the art and would be useful in the practice of the present invention.

Orally acceptable carriers include any vehicle or delivery means, including a combination of a fluid or solid carrier with a delivery device, that is capable of delivering a free radical scavenging precursor compound, such as those compounds of formula I, to one or more tissue surfaces in the oral cavity. Orally acceptable carriers include dosage forms such as toothpastes (dentifrices), gels, mouthwashes, rinses, chewing gums, lozenges, floss, interdental stimulating sticks, denture adhesives, buccal patches, tooth balms, dental tray-administered gels or pastes, sprays, chewable objects (such as an animal chew toy comprising rawhide as a carrier), food or feed coatings, topical dressings, or tooth varnishes. The orally acceptable carrier may also be administered to the oral cavity by means of or as part of an assembly or device, such as a dental tray, plastic strip, buccal patch, gingival retraction cord, or curable restorative material (such as a temporary cement or free-radically polymerized tooth composite). Oral acceptable carriers or delivery means for ascorbyl phosphate, other than those listed here, are known to those skilled in the art and would be considered to have utility in the practice of this invention.

Orally acceptable carrier ingredients include water-soluble fluids, water-soluble solids, non-water soluble fluids, non-water soluble solids, humectants, thickeners, abrasives, surfactants (anionic, cationic, non-ionic and zwitterionic), sweeteners, flavorants, colorants (dyes and pigments), abrasives, stabilizers, polymeric film-forming agents, gum base,

Water-soluble fluids include water, glycerin, propylene glycol, polyethylene glycol (with a molecular weight less than about 1000), butylene glycol, ethyl alcohol, and mixtures thereof.

Water-soluble solids include sorbitol, xylitol, maltitol, mannitol, other polyhydric alcohols, polyethylene glycol (with a molecular weight greater than about 1000), and mixtures thereof.

Non-water soluble fluids include mineral oils, vegetable oils, natural or synthetically derived fluid esters, and mixtures thereof.

Non-water soluble solids include petrolatum, waxes (natural and synthetic), polybutylene, low molecular weight waxy polymers, and mixtures thereof.

Humectants include glycerin, propylene glycol, polyethylene glycol (molecular weights between about 200 and about 8000), butylene glycol, sorbitol, xylitol, maltitol, mannitol, other polyhydric alcohols, and mixtures thereof. Some humectants may also serve as water-soluble solids within the orally acceptable carrier.

Thickeners include carboxypolymethylene, acrylate polymers and copolymers, carboxymethylcellulose (preferably the sodium salt), hydroxypropyl cellulose, hydroxyethyl cellulose, xanthan gum, poly(maleic anhydride/methyl vinyl ether), poly(vinyl pyrollidone), vinyl pyrollidone copolymers, poly(vinyl acetate), vinyl acetate copolymers, hydrated silica, fumed silica, magnesium aluminum silicate, and other polymeric or inorganic thickeners known in the art. Salts of thickeners listed above are also contemplated. Mixtures of the above thickeners may also be advantageously employed.

Surfactants (surface active agents, also called, depending on their intended function, detergents, foaming agents, dispersants, solubilizers, or emulsifiers) include sodium lauryl sulfate, sodium lauroyl sarcosinate, sodium methyl cocoyl taurate, sodium dodecyl benzenesulfonate, sodium lauryl sulfoacetate, poloxamers (sold under trade name Pluronic), polyoxyethylene sorbitan esters (sold under trade name Tween), fatty alcohol ethoxylates, polyethylene oxide condensates of alkyl phenols, cocoamidopropylbetaine, and mixtures thereof.

Sweeteners include sugars, sugar alcohols, saccharin, potassium acesulfame, aspartame, sucralose, and mixtures thereof.

Flavorants include oil of wintergreen, oil of peppermint, oil of spearmint, methyl salicylate, menthol, thymol, anethole, oil of clove, eucalyptol, eugenol, oil of cinnamon, vanillin, and mixtures thereof.

Colorants include orally acceptable FD&C and D&C dyes and lakes, zinc oxide, titanium dioxide, natural and synthetic colorants, and mixtures thereof.

Abrasives include silica (precipitated and gel), dicalcium phosphate dihydrate, dicalcium phosphate anhydrous, hydrated alumina, insoluble sodium polyrnetaphosphate, calcium carbonate, calcium pyrophosphate, tricalcium phosphate, and mixtures thereof. If an abrasive is desired, silica abrasives are preferred, due to their inertness to many active therapeutic ingredients, such as sources of fluoride ion.

Stabilizers include chelating agents (such as EDTA, bisphosphonates, citric acid, and gluconic acid), preservatives (such as benzoic acid and its salts, methyl paraben, propyl paraben, potassium sorbate, and mixtures thereof) and other ingredients that can improve the shelf life or activity of a particular active ingredient and/or formulation.

The inventive compositions may also include a source of calcium ions in order to encourage the precipitation of one or more forms of calcium phosphate during use. Alternatively, the source of calcium ions may be saliva or saliva-coated surfaces in the oral cavity. Compositions containing both an ascorbyl phosphate ester and a calcium ion source may have utility in the remineralization of tooth enamel, and may be useful in the reversal of such early stage oral hard tissue disease processes such as primary root caries lesions. In one embodiment, the calcium ion source may be an ingredient of a one part composition, that is, formulated into a single mixture that also contains the ascorbyl phosphate and packaged as a one part mixture. In another embodiment, the calcium ion source may also be an ingredient of one part of a two part composition, that is, formulated into a first mixture that is separate and distinct from a second mixture that contains ascorbyl phosphate. In this particular embodiment, the two mixtures are packaged separately from one another to avoid mixing or contact with one another until the point of use or application to the oral cavity. A particularly useful package for this embodiment is a dual-chambered syringe with an attached static mixer assembly, whereby a first calcium ion containing mixture is placed in one of the two chambers and a second ascorbyl phosphate containing mixture is placed in the other of the two chambers, and whereby external pressure applied to a syringe plunger forces the two mixtures into the static mixer assembly. The two mixtures thus forced through the static mixer assembly combine and mix as they pass through or past the baffles of the static mixer assembly to become a single mixture containing both the calcium ions and the ascorbyl phosphate. The mixture emerges from an opening in the static mixer assembly distal to the opening into which the two separate mixtures first entered the static mixer assembly. Manual mixing of the two mixtures placed on a surface and agitated by means of a spatula or similar tool is also contemplated. In yet another embodiment, the calcium ion source may be found in saliva or on an oral cavity surface, whereby the mixing of an ascorbyl phosphate mixture with calcium ions occurs in situ, that is, on one or more oral cavity surfaces.

Other auxiliary or supplemental active ingredients, many of which are known to those skilled in the art, may also be included in the ascorbyl phosphate compositions, and are described in more detail below. Such auxiliary ingredients may provide additional, and in some cases complimentary, biological or therapeutic activity to the ascorbyl phosphate.

### Anticaries Agents

One or more anticaries agents, in particular fluoride containing or releasing compounds, may be advantageously included in the ascorbyl phosphate compositions. Useful anticaries agents include sodium fluoride, sodium monofluorophosphate, stannous fluoride, amine fluorides, and other fluoride containing compounds capable of increasing the resistance of mineralized tissues in the oral cavity to caries formation (tooth decay). Anticaries agents may be included in the ascorbyl phosphate compositions at concentrations of from about 0.1% to about 4% by weight of the composition, and preferably from about 0.2% by weight to about 0.8% by weight of the composition.

### Tartar Control Compounds

One or more tartar control agents, also known as anticalculus agents, may be employed in the ascorbyl phosphate compositions to assist in the reduction or prevention of tartar formation on the teeth. Useful tartar control agents include the sodium and potassium salts of pyrophosphate, tripolyphosphate, and polyphosphates, as well as other calcium chelating or calcium sequestering agents known in the art. Tartar control compounds may be included in the ascorbyl phosphate compositions at concentrations between about 0.1% and about 10% by weight of the composition, and preferably between about 1% and about 4%, by weight of the composition.

### Antimicrobial Agents

In order to reduce or eliminate microorganisms responsible for oral diseases such as gingivitis, periodontitis, caries, and halitosis, an antimicrobial agent may be included in the inventive ascorbyl phosphate compositions. Such compounds are well known in the art, and include triclosan, chlorhexidine (and its salts), cetylpyridinium chloride, and essential oils including menthol, eucalyptol, thymol and methyl salicylate. Antimicrobial agents may be included in the ascorbyl phosphate compositions at concentrations between about 0.01% and about 2% by weight of the composition, and preferably between about 0.1% and about 1%, by weight of the composition.

### Tooth Desensitizing Agents

While the ascorbyl phosphate compounds described herein may also exhibit activity as tooth desensitizers, due to the release of phosphate ions into salivary solution upon contact with the oral cavity (thus encouraging tooth remineralization resulting from the precipitation of calcium phosphate at the tooth surface), it may be advantageous to add a supplementary tooth desensitizer such as potassium nitrate, potassium citrate, or strontium chloride hexahydrate in order to further alleviate tooth sensitivity. Such supplementary tooth desensitizers may be included in the ascorbyl phosphate compositions at a concentration of from about 0.1% by weight to about 10% by weight of the composition. Potassium nitrate is the preferred supplementary tooth desensitizer and is included in the composition at a concentration of from about 3% to about 6% by weight of the composition, and preferably at about 5% by weight of the composition.

The ascorbyl phosphate compositions, together with any auxiliary active ingredients, may be in the form of (or delivered to the oral cavity by means of) a toothpaste (dentifrice), gel, mouthwash, chewing gum, lozenge, floss, interdental stimulating stick, denture adhesive, buccal patch, tooth balm, dental tray-administered gel or paste, spray, chewable object (such as an animal chew toy comprising rawhide as a carrier), food or feed coatings, topical dressing, or tooth varnish.

Application of the ascorbyl phosphate compositions depends upon the nature of the carrier, but in general may be accomplished by brushing, rinsing, spraying, chewing, swabbing, adhering or otherwise applying said compositions to one or more oral tissue surfaces. Application may also be made by placing an ascorbyl phosphate ester containing composition, for instance a gel, into a dental tray and attaching the tray to the maxillary (upper) and/or mandibular (lower) arch of teeth so that the teeth make contact with the gel inside the tray.

The following examples are set forth as representative of the present invention. These examples are not to be construed as limiting the scope of the invention as these and other equivalent embodiments will be apparent in view of the present disclosure, tables, and accompanying claims.

### Example 1

According to a certain embodiment of the present invention, a free radical scavenging precursor compound, i.e. sodium ascorbyl-2-monophosphate, is formulated into a mouthwash including the ingredients set forth below.

| Ingredient | Percent (w/w) |
|---|---|
| Deionized water | 86.190 |
| Glycerin | 7.500 |
| Sodium tripolyphosphate | 3.000 |
| Polyethylene glycol 8000 | 1.000 |
| Sodium saccharin | 0.060 |
| Sodium benzoate | 0.500 |
| Sodium ascorbyl-2-monophosphate | 1.000 |
| PEG-60 hydrogenated castor oil | 0.600 |
| Flavor | 0.150 |
| Total | 100.000 |

The above composition had a pH of 8.86, a specific gravity of 1.058, and a refractive index of 1.3530.

### Example 2

According to certain embodiments of the present invention, a toothpaste including a free radical precursor compound, i.e. sodium ascorbyl-2-monophosphate, is formulated to include the following ingredients.

| Ingredient | Percent (w/w) |
|---|---|
| Deionized water | 17.298 |
| Sodium ascorbyl-2-monphosphate | 0.500 |
| Sodium benzoate | 0.500 |
| Sodium fluoride | 0.240 |
| Titanium dioxide | 1.200 |
| Sodium saccharin | 0.400 |
| Sorbitol (70% solution) | 34.562 |
| Glycerin | 18.000 |
| Cellulose gum | 1.000 |
| Hydrated silica (abrasive) | 17.500 |
| Hydrated silica (thickener) | 7.000 |
| Sodium lauryl sulfate | 1.000 |
| Flavor | 0.800 |
| Total | 100.0000 |

The following chart is a summary of the approximate range of concentrations (on a weight percent basis) for components of mouthwash and toothpaste compositions containing the ascorbyl phosphate compound. The term ascorbyl phosphate includes one or more phosphate esters of ascorbic acid, either alone or in combination. The term ascorbyl phosphate shall also include any corresponding inorganic or organic salts of phosphate esters of ascorbic acid. A preferred ascorbyl phosphate is ascorbyl-2-monophosphate and a more preferred ascorbyl phosphate is the trisodium salt of ascorbyl-2-monophosphate, also known simply as sodium ascorbyl phosphate.

TABLE 1

| Component | Mouthwash | Toothpaste/Gel |
|---|---|---|
| Water | 46-99.9% | 0-99.89% |
| Ascorbyl-2-phosphate | 0.01-10% ¹⁰ | 0.01-10% |
| Fluoride ion (F-) source | 0-4% | 0-4% |
| Auxiliary active ingredient | 0-10% | 0-10% |
| Preservative | 0-3% | 0-3% |
| Humectant | 0-30% | 0-70% |
| Artificial sweetener | 0-1% | 0-2% |
| Thickener/binder | 0-5% | 0.1-20% |
| Surfactant | 0-5% | 0-5% |
| Abrasive | 0% | 0-60% |
| Pigment/Dye | 0-0.2% | 0-5% |
| Flavorant | 0-2% | 0-3% |
| pH range | 5.5-10.0 | 5.5-10.0 |

Percentages in Table 1 above are in terms of weight percent, as based upon the total weight of the formulation.

Compounds of formula I may also be added to other types of oral care compositions, as well as certain types of foodstuffs, including, but not limited to, chewing gum, dental floss, tooth whitening gels and pastes, breath sprays, buccal patches, medicament delivery strips, and lozenges. Furthermore, any device or carrier for delivery of a compound of formula I to hard and/or soft tissue surfaces in the oral cavity is contemplated have utility as a delivery system or device for the compositions, and in the practice of the methods, of the present invention.

Also contemplated is the inclusion of a phosphatase enzyme inhibitor, such as a fluoride ion source, in the compositions of the present invention. Other auxiliary oral care ingredients, such as those employed for tartar control, tooth bleaching or whitening, halitosis elimination or prevention, and microbial control, may also be included. Also, one or more ingredients for retaining the compound of formula I on hard or soft tissue surfaces for extended periods of time (for instance, in excess of one hour) are contemplated. Such ingredients may include a polymer, said polymer either physically or ionically binding the compound of formula I in a fashion so as to keep it in intimate or close proximity to the tooth or oral mucosal surface.

The compositions of the present invention may be prepared and packaged for use as one part compositions (whereby no mixing of components is necessary prior to applying the composition to the oral cavity). Suitable packages include syringes, tubes, bottles, jars, and unit dose packages, to name a few.

Alternatively, two part compositions (whereby two separately packaged components are combined to form a single component mixture just prior to application to the oral cavity) may be utilized. Two part compositions may be packaged by placing one of the components of the system in one chamber of a dual chambered syringe, and the other component in the other chamber of the dual chambered syringe. Mixing of the two components is accomplished by applying pressure to a syringe plunger that forces the contents of both chambers into and through a series of mixing elements that are attached to one end of the dual chambered syringe. Such a mixing element assembly is known in the art as a static mixer. The two components thus become mixed as they are forced into one end of the static mixer, through the static mixer, and finally out the other end of the static mixer assembly. The mixture that emerges from the end of the static mixer assembly is preferably applied directly to the oral cavity, rather than being stored for an extended period of time.

In addition, two or multi-part systems may be applied in sequence, whereby one separately packaged component is applied to a surface of the oral cavity, followed by the application (to the same oral cavity surface) of a second separately packaged component to the oral cavity, etc. Mixing of the two or more components is thus accomplished in situ, rather than prior to application.

### Example 3

### Method of Preventing Tooth Sensitivity Associated with Peroxide Tooth Whitening

Most tooth whitening compositions that are capable of eliminating or reducing both extrinsic and intrinsic tooth staining contain an oxidizing compound. Typically, the oxidizing compound is either hydrogen peroxide, or a precursor to hydrogen peroxide, such as carbamide peroxide, sodium perborate, sodium percarbonate, and others. It is known that hydrogen peroxide is able to penetrate through intact enamel and dentin (and more easily through cracks in enamel or exposed root surfaces), thus reaching vital pulp tissue within 15 minutes from initial contact of the peroxide tooth whitening composition on the tooth surface. It is speculated that the presence of peroxide in the pulp chamber is one of the major contributors to tooth sensitivity associated with such tooth whitening procedures.

A particularly useful application of the inventive ascorbyl phosphate compositions is for alleviating or preventing the tooth sensitivity often associated with the use of peroxide-containing tooth whitening compositions. Upon contact with a tooth surface that has been treated with a peroxide-containing tooth whitening composition, the ascorbyl phosphate compositions provide a source of ascorbic acid upon hydrolysis by phosphatase enzymes present in the oral cavity. Ascorbic acid is known to be a powerful free-radical scavenger. While not wishing to be bound by any particular theory, release of ascorbic acid from ascorbyl phosphate applied to the oral cavity may reduce the likelihood of pulp tissue damage by scavenging free-radical degradation products of hydrogen peroxide, such as the hydroxyl radical (.OH) and the perhydroxyl radical (.OOH). An additional benefit may be obtained after hydrolysis of the ascorbyl phosphate molecule, in that free phosphate ion is released into salivary solution and at the tooth surface, thus creating conditions that are highly conducive to formation (typically by precipitation) of calcium phosphate crystals within the dentinal tubules. Blockage of the dentinal tubules is known to decrease tooth sensitivity by reducing the likelihood of fluid movement within the tubules caused by external stimuli, such as heat, cold, and contact with hygroscopic foodstuffs.

A preferred method of reducing or eliminating the tooth sensitivity associated with peroxide-based tooth whitening procedures comprises the sequential steps of
1. Contacting a tooth surface or tooth surfaces with a peroxide-containing tooth whitening composition for a period of time in order to effect tooth whitening,
2. Contacting the same tooth surface or tooth surfaces with a composition comprising a compound of formula I, such as an ascorbyl phosphate
   Optionally, the ascorbyl phosphate compositions of the above method may contain other ingredients commonly employed in oral care formulations, such as humectants, thickeners, preservatives, foaming agents, solubilizers, adherence-enhancing agents, phosphatase enzyme inhibitors, antimicrobial agents, anticaries agents, tartar control agents, tooth desensitizing agents, sweeteners, and flavorants.

The carrier or dissemination means for the ascorbyl phosphate in the method above can be a liquid, gel, paste, cream, stick, chewing gum, or any other oral care vehicle as would be well known to those skilled in the art. The compositions of the above method may be applied or positioned into close proximity with an oral cavity surface by rinsing, brushing, spraying, or chewing. An oral cavity surface may be the surface of a tooth, the oral mucosal, the tongue, or even a surface temporarily exposed during a dental surgical procedure, such as a root canal or cavity excavation. Another means of applying the compositions of the above method onto an oral cavity surface is by placing the composition in a dental tray, on a strip, or on a patch, and then placing said dental tray, strip or patch in the oral cavity, preferably in direct contact with the oral cavity surfaces to be treated.

### Example 4

### Denture Adhesive Comprising a Sodium/Calcium Mixed Salt of Ascorbyl-2-Phosphate

Useful compositions have been prepared that demonstrate antioxidant activity when used as a denture adhesive to temporarily affix a denture to an oral mucosal surface. One example of such a denture adhesive is provided in the table below.

| | |
|---|---|
| Petrolatum | 31.259 |
| Mineral Oil | 14.271 |
| Ascorbyl-2-phosphate, Na/Ca mixed salt (Rovimix Stay-C 35) | 2.000 |
| Hydrated silica | 1.833 |
| Poly(methyl vinyl ether/maleic anhydride), Na/Ca mixed salt | 32.285 |
| Cellulose Gum | 18.052 |
| Sodium Saccharin | 0.100 |
| Methyl Paraben | 0.100 |
| Propyl Paraben | 0.100 |
| Total | 100.000 |

1-46. (canceled)

47. A method for reducing tooth sensitivity comprising:
contacting a tooth surface or tooth surfaces with an oral care composition comprising
(a) an orally acceptable carrier; and
(b) an ascorbyl-2-phosphate compound having the following structure, or a sodium or potassium salt thereof, 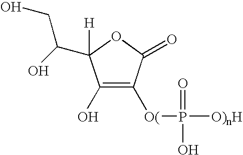
wherein n is between 1 and 10.

48. The method of claim 47 wherein the oral care composition contacts the tooth or tooth surfaces for 5 to 60 minutes.

49. The method of claim 47 further comprising contacting a tooth surface or tooth surfaces with a peroxide-containing tooth whitening composition for a period of time in order to effect tooth whitening prior to contacting with an oral care composition.

50. The method of claim 49 wherein the oral care composition contacts the tooth or tooth surfaces for 5 to 60 minutes.

51. A method for counteracting tooth decay or assisting in the regenerative process of periodontal tissues comprising:
contacting a tooth surfaces or tooth surfaces with an oral care composition comprising
(a) an orally acceptable carrier; and
(b) an ascorbyl-2-phosphate compound having the following structure, or a sodium or potassium salt thereof, 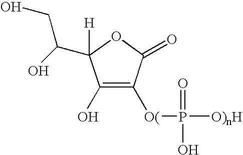
wherein n is between 1 and 10.

52. The method of claim 51 wherein the oral care composition contacts the tooth or tooth surfaces for 5 to 60 minutes.

53. A method for the prevention of tooth stain accumulation comprising:
contacting a tooth surfaces or tooth surfaces with an oral care composition comprising
(a) an orally acceptable carrier; and
(b) an ascorbyl-2-phosphate compound having the following structure, or a sodium or potassium salt thereof, 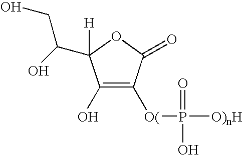
wherein n is between 1 and 10.

54. The method of claim 51 wherein the oral care composition contacts the tooth or tooth surfaces for 5 to 60 minutes.

55-63. (canceled)
